(19) United States
(12) Patent Application Publication
(10) Pub. No.: US 2003/0082560 A1
(43) Pub. Date: May 1, 2003

(54) Method of making interactive protein arrays

(76) Inventor: Yingjian Wang, New Britain, CT (US)

Correspondence Address: Yingjian Wang, 1 Peace Court, New Britain, CT, 06051, US

(21) Appl. No.: 10/055,838

(22) Filed: Oct. 29, 2001

(51) Int. Cl. C12Q 1/68; B05D 3/00; G01N 33/53; G01N 33/542; C12M 1/34

(52) U.S. Cl. 435/6; 435/7.9; 435/287.2; 427/2.11

(57) ABSTRACT

A device for biomedical research contains a flat solid support and a plurality of proteins, with the proteins immobilized on the support at pre-determined positions. The proteins immobilized at each position can be different but share a common property. An interactive protein array is produced when proteins that interact with a ligand are immobilized at a position and proteins interact with another ligand are immobilized at another position on the flat support. The interactive protein arrays are produced either by isolating interacting proteins sequentially or simultaneously. Chemical covalent cross-linking is used to stabilize the protein complexes on the array and to immobilize the proteins on the flat support. The devices are used in studying proteins, particularly in identifying protein-protein interactions and their modulators.

### BACKGROUND

The field of this invention relates to methods for detecting proteins, in particular, for detecting protein-protein interactions.

Proteins are the major component of cells. One important mechanism by which proteins carry out their functions is through interacting with other molecules. Interactions between hormones and their protein receptors on the cell surfaces are the key steps in cells' response to environmental changes. Inside the cells, protein-protein interactions are the principle mechanisms in signal transduction and regulation of enzymatic activities. In the nuclei, binding of transcriptional factors at the promoter region of a gene is critical for the expression of that gene. Therefore, identification of protein interactions, especially protein-protein interactions, is the key for the understanding of cells and is of immense value to biomedical research.

Because of the importance, a significant amount of research effort is devoted to the studies of protein-protein interactions and their physiological functions. Currently, there are several methods to detect protein-protein interactions (for a review, see Phizicky and Fields, Microbiological Reviews, p94-123, March 1995). Among them, co-immunoprecipitation, yeast two-hybrid screening and phage display library screening are the most commonly used methods. In co-immunoprecipitation, a protein of interest is precipitated with its specific antibody. Other proteins that interact with the protein of interest will be also precipitated and can be identified by blotting with a specific antibody or by purification and sequencing. A related method is affinity purification, in which a protein of interest is immobilized on a solid support and used to isolate any interacting proteins (Phizicky and Fields, Microbiological Reviews, p94-123, March 1995). The current methods of co-immunoprecipitation and affinity purification have some limitations. For example, it is difficult to apply them in large-scale identification of protein-protein interactions. Yeast two-hybrid screening is a genetic method for detecting protein-protein interactions (Fields and Song, U.S. Pat. No. 5,283,173, 1994; Fields and Song, 1989, Nature, 340:245-246). Although a single yeast two-hybrid screening assay can detect many protein interactions, the assay is time-consuming and prone to false positive results. Moreover, the yeast two-hybrid method is unable to detect many protein-protein interactions that occur only in the presence of additional cellular factors or after posttranslational modifications, which may not be present or occur in yeast. Phage display library screening of protein-protein interactions suffers similar limitations (Smith, 1985, Science 228:1315-1317). All of these methods are mainly for use in laboratory research and it is difficult to adapt them to large-scale screening. Therefore, methods that could be used for high throughput screening of protein-protein interactions are in great demand.

As more and more biological reagents have become available, such as numerous deoxyribonucleic acid (DNA) clones and recombinant proteins, several methods have been developed that make use of them. DNA Array and Protein Array are two recently developed technologies that have been applied in high-throughput studies of many biological problems. In a DNA array, many DNA clones are immobilized on a flat solid support, each one at a predetermined position so that it can be identified later by this unique position on the support. Two types of DNA arrays are currently available: cDNA array and oligo array. In a cDNA array, a large number of complementary DNA (cDNA) are prepared and each of them is deposited and immobilized on a solid support (Brown et al., U.S. Pat. No. 5,807,522). In an oligo array (such as a DNA chip manufactured by Affymatrix), oligo nucleotides are either directly synthesized on the supports (Pirrung, et al., U.S. Pat. No. 5,143,854, 1992; Fodor, et al., Science, 251: 767-773, 1991; Southern, et al., Genomics 13:1008-1017, 1992) or synthesized first and then immobilized on a support. Similarly, in a protein array, many different proteins are immobilized on a solid support, each protein at a predetermined position so that it can be identified subsequently by its unique position on the support. Two types of protein arrays are particularly useful: antibody arrays and recombinant protein arrays, which contain a plurality of antibodies or recombinant proteins immobilized on solid supports, respectively (Wang et al. 2000; Mol. Cell Biol. 20(13), 4505-12; Ge, 2000 Ge, H. Nucleic Acids Research, Vol. 28:e3.). In addition, arrays of cells, tissues, lipids, polymers, drugs, or chemical substances can also be fabricated for large-scale screening assays (Kononen J, et al., Nature Medicine, 4:844-7, 1998).

DNA and protein arrays have found a wide variety of applications in basic biomedical research, clinical diagnostics, and drug development. DNA arrays are used for hybridization assays, including monitoring of gene expression (Schena et al., 1995, Science 270:467-470; DeRisi et al., 1996, Nature Genetics 14:457-460), genetic and physical mapping of genomes, genetic diagnosis, genotyping of organisms, and distribution of biological reagents to researchers (see U.S. Pat. No. 5,807,522). DNA arrays are also used to get nucleotide sequence information, including mutation detection, polymorphism analysis, and DNA sequencing (Hacia, Nature Genetics Volume 21, supplement, p42-47, 1999). Another application of DNA arrays is to study the interactions between DNA and proteins, such as the identification of a DNA sequence to which a protein binds (Bulyk et al. Nature Biotechnology, 17:573-577, 1999).

Protein arrays have been used to capture and separate a plurality of proteins of interest from a protein mixture (Wang et al. 2000; Mol. Cell Biol. 20(13), 4505-12; Ge, 2000 Ge, H. Nucleic Acids Research, Vol. 28:e3). After capture and separation, the proteins of interest can be further characterized. Therefore, protein array technology makes it possible to study the properties of a large number of proteins in a single assay. Specifically, protein arrays have been applied in studying protein expression patterns, protein posttranslational modifications, and protein-protein interactions. To study the protein expression pattern of a protein sample, an antibody array is incubated with the sample to allow binding between antibodies and antigens. After incubation, unbound or non-specific bound proteins are removed by several washes. Proteins specifically bound at each position of the array are then detected. As antibodies are immobilized in a predetermined order, the identity of the protein captured at each position is therefore known. Measurement of protein quantities at all positions of the array thus reflects the protein expression pattern in the sample.

Protein arrays can be applied in studying protein posttranslational modifications such as phosphorylation, glycosylation, lipidation, and ubiquitination. To do so, antibody arrays are used to capture cellular proteins. The phosphorylation of the proteins captured on the array can be revealed if the proteins are metabolically labeled with P-32 in vivo. Alternatively, the phosphorylation can be detected by antibodies against phosphorylated amino acids. Similarly, the glycosylation and lipidation of the many proteins captured on the array can be studied either by labeling proteins with radioactive precursors or by using molecules that specifically recognize the carbohydrate or lipid moieties of proteins. One family of molecules that recognizes the carbohydrate moieties of glycoproteins is lectins. To detect protein ubiquitination, antibodies specific for ubiquitin can be used.

Another important application of protein arrays is to examine protein-protein interactions. If a protein interacts with other proteins in a protein sample, when the protein is captured by its antibodies immobilized on an antibody array, other proteins may also be tethered to the same position due to protein-protein interactions. Therefore, to examine what proteins interact with a protein of interest, a protein mixture (e.g., cell lysate) is made under such conditions that protein-protein interactions are preserved. After incubation of the protein mixture with an antibody array, the protein of interest will be captured to the position where its interacting protein(s) is captured. By localizing the position of the interested protein, the identity of its interacting protein is known because the identity of the antibody immobilized at each position is predetermined. The protein of interest can be localized by several methods including the use of a specific antibody. The protein of interest can be expressed as a fusion protein with a tag and can then be detected by the tag's specific property. To use a recombinant protein array to detect protein-protein interactions, the array is first incubated with a protein of interest (a purified protein or a cellular protein among thousands of other proteins). The protein of interest is then detected on the array to find out which recombinant protein interacts with it.

Protein arrays overcome some of the limitations that other methods have in studying protein-protein interactions. Compared to yeast two-hybrid method, which is time-consuming and prone to false positive results, the protein array assay has the advantages of consuming far less time (an assay can be usually performed in just one day) and being dependent on fewer factors (the quality of assay is mainly determined by the specificities of the antibodies or the proteins used). Moreover, many protein-protein interactions only occur in the presence of additional cellular factors or after posttranslational modifications, which may not be present in yeast. Therefore, the yeast two-hybrid method fails to identify many important protein-protein interactions that only take place in mammalian cells. Phage display screening of protein-protein interactions suffers similar limitations. In contrast, Protein Array technology can directly detect protein-protein interactions under normal cellular conditions. Most importantly, unlike co-immunoprecipitation, affinity purification, yeast two-hybrid screening and phage display, protein array can be employed easily in high-throughput screening of protein-protein interactions. Similar to DNA arrays, protein arrays also make a large number of biological reagents, such as antibodies and recombinant proteins, readily available to researchers, therefore, a researcher can get an antibody array containing hundreds of different antibodies at an affordable price. Several commercial products based on protein array technology have been developed for studying protein expressions, protein posttranslational modifications and protein-protein interactions.

Powerful as it is in studying protein-protein interactions, current protein array methods have some limitations. First, protein-protein interactions are susceptible to disruption during assays. For example, in order to detect one protein-protein interaction with an antibody array, several interactions (the interactions between immobilized antibodies and their antigens, the antigens and their interacting proteins, the interacting proteins and their antibodies) have to be maintained during the assay. However, protein-protein interactions are reversible, and these interactions—especially the interactions between antibodies and their antigens, antigens and their interacting proteins—have to endure long incubation times and extensive washes, which may disrupt a significant portion of the interactions and thus decrease the final signals. This problem is particularly severe for weak protein-protein interactions.

Second, most protein arrays are not re-usable. After probing a protein on a protein array, in order to use the same array again, proteins bound to the array, especially the detecting antibodies, would have to be removed. A typical procedure to remove antibodies from antigens is treatment with 5% SDS, 100 mM beta-mercaptanol ethanol for 30 minutes at 50° C. However, such treatment will render an antibody array unusable because it denatures the antibodies so that they can no longer interact with antigens. Recombinant protein arrays suffer similar problems. Even if a protein array is reusable after stripping off previously probed antibody, because the stripping also removes any binding partners, the array has to be incubated with a protein sample again to capture binding partners and then probed with other antibodies. Thus, additional materials and work are incurred for probing each additional protein.

Third, in many immunochemical methods such as Western blotting and immunostaining, after binding of a primary antibody to an antigen, the primary antibody is then usually detected with a secondary antibody that is conjugated with either an enzyme or a fluorescent molecule. Although enzyme- and fluorescent-conjugated primary antibody can be used, a conjugated secondary antibody is usually preferred because a conjugated secondary antibody can be used in combination with many different primary antibodies. Because antibody arrays usually have both polyclonal and monoclonal antibodies immobilized on them, a problem in detection is that when an antibody is used to identify the protein of interest, an enzyme or fluorescent labeled secondary antibody cannot be used because the secondary antibodies will bind the detecting primary antibodies and the capturing antibodies immobilized on the array indiscriminately. Therefore, the primary antibody usually has to be conjugated and for each antibody, such conjugation has to be prepared.

Studying DNA-protein interactions using DNA arrays suffers similar limitations. For example, the interactions between DNA and proteins may be disrupted during the assay and it is difficult to use the same array more than once.

### PURPOSE OF THE INVENTION

It is an object of the present invention to describe a new improved protein array which has proteins with a common property immobilized at a position of the array. Particularly, it is an object of the present invention to describe an interactive protein array, which at each position has proteins interacting with a specific ligand immobilized.

It is also an object of the invention to describe a method to make improved protein arrays. In particular the invention teaches the use of cross-linking and array transfer in making the protein arrays.

It is a further object of the present invention to teach a method to use the improved protein arrays in detecting proteins and their properties, particularly in detecting protein-protein interactions and protein posttranslational modifications. Methods are also described to screen modulators of protein-protein interactions.

### SUMMARY OF THE INVENTION

The present invention describes a new type of protein arrays. Different from the currently available protein arrays, the new protein arrays have proteins with a common property immobilized at a position on the array. One particular type of array is an Interactive Protein Array that at each position has proteins interacting with a specific ligand immobilized. The invention describes the method to make the new protein arrays, especially the interactive protein arrays. In particular novel cross-linking and transfer methods are described in making the protein arrays. The invention also pertains to the method for using the improved protein arrays in detecting protein-protein interactions. In another embodiment of the present invention, interactive protein arrays are used to screen protein-protein interactions in a protein sample. Methods are also described to screen modulators of protein-protein interactions.

### BRIEF DESCRIPTION OF THE DRAWINGS

FIG. 1 shows an example of increasing Western blotting sensitivity by cross-linking.

FIG. 2 shows an example of increasing assay sensitivity by cross-linking in detecting protein-protein interactions using antibody arrays.

FIG. 3 shows an example of repeated screening of protein-protein interactions using an interactive antibody array made by cross-linking.

FIG. 4 shows an example of making interactive protein arrays by transferring; and detecting proteins on the arrays.

### DESCRIPTION OF THE PREFERRED METHODS

As used herein, the term “One protein” usually refers to a collection of polypeptides which have the same amino acid sequence. However, in many cases, “one protein” may also refer to a collection of polypeptides which may have different amino acid sequences but the sequence differences have no effect on the purpose of the assay and no attempt is made to distinguish them. For example, a polyclonal antibody refers to a collection of immunoglobulins, which have a variety of different polypeptide chains and they may also recognize different epitopes on the same or different proteins. But since no attempt is made to distinguish them, for practical purpose they may still be regarded as one antibody.

As used herein, the term “Protein of interest” refers to the protein to be examined in an assay. It is usually a cellular protein but can also be an antibody, a recombinant protein, or synthesized peptides.

As used herein, the term “Protein-protein interaction” refers to the close and stable association between proteins. It usually involves the formation of non-covalent chemical bonds such as hydrogen bonds. Direct interaction means that two proteins involved have close contact and form chemical bonds between them. Indirect interaction between two proteins occurs when they do not interact directly with each other but are bound together through interacting with other proteins which in turn interact directly or indirectly with each other.

As used herein, the term “Immobilization” refers to the fixation of molecules on a solid support so that they cannot move freely. The fixation can be covalent, such as by covalently cross-linking the molecules on the solid support. Or alternatively, molecules can be fixed on the support by non-covalent binding, such as immobilization of proteins on a nitrocellulose membrane. Immobilization can also be through specific molecules. For example, a first protein can be immobilized on a support via covalent bond; a second protein can then be immobilized at the same position on the support via specific interaction with the first protein.

As used herein, the term “Interactive protein array” refers to a protein array which has a plurality of positions, each position containing proteins that interact with a ligand or a set of ligands.

As used herein, the term “Ligand” refers to an agent that can bind one or more proteins. It can be any molecule selected from, but not limited to, antibodies, recombinant proteins, synthesized peptides, DNA, RNA, carbohydrates, and organic molecules.

In a typical protein array (an antibody array or a recombinant protein array), one protein is usually immobilized at a position of the array. In the case of an antibody array, although many different antibodies against different epitopes of a protein can be present in a polyclonal antibody, the whole collection of that polyclonal antibody is usually still regarded as one protein. Even several antibodies from different sources but all against the same protein are immobilized at the same position, they may still be regarded as one protein because it is not necessary to distinguish them and no attempt is made to do so in the assay.

A more general protein array would be that instead of just one protein, a group of proteins with a common property are immobilized at each position on the solid support. Thus, proteins at one position have one property; proteins at another position have another property; and so on. Any property can be used to group proteins, as long as methods are available to prepare each group of the proteins. Some of the protein properties that can be used are molecular weight, isoelectric point, density, specific chemical group on the proteins, solubility in a solvent, and subcellular localization. Methods to group proteins according to these properties are known to persons skilled in the art. Since most proteins form complexes in cells and the molecular weights and sizes of the complexes are different, protein complexes can be separated according to molecular weight. Then a protein array can be made so that the proteins at each position of the array are components of protein complexes (each complex contains one or more proteins) which have molecular weights within a certain range. For example, proteins at the first position are components of protein complexes with a molecular weight of 0 to 1 million, proteins at the second position belong to complexes with a molecular weight of 1 to 2 million, proteins at the third position belong to complexes with a molecular weight of 2 to 3 million, and so on. To make such an array, proteins were prepared from cells or tissues under conditions to maintain intact protein complexes. Then the proteins were separated into different fractions, e.g., by chromatography, so that protein complexes in each fraction have a specific molecular weight. The proteins in each fraction whose molecular weights can be estimated are then immobilized at a specific position on a solid support thus to form an array. This array is useful in studying protein activities and properties.

In a general protein array, each set of proteins immobilized at a position may have one or more proteins. When more than one protein is immobilized at a position, these proteins are treated as different proteins because one or more members of them may be identified and thus distinguished from the other proteins on the same position. This is different from the case of a typical antibody array where the polyclonal antibodies against one antigen are immobilized at one position, and although polyclonal antibodies contain more than one type of peptide, they are not distinguished in the assay. Of course, the exact identities of the proteins immobilized at each position in a general protein array may not be known.

One type of protein array of particular interest to the present invention is the Interactive Protein Array. In a standard interactive protein array, the proteins at a position interact with one ligand, and proteins at another position interact with another ligand. In other words, in an interactive protein array, proteins interacting with a particular ligand are immobilized at one position of the support and proteins interacting with another ligand are immobilized at another position, and so on. In many situations, the identities of the proteins immobilized at a position may be unknown. For example, when a specific known ligand is used to isolate its interacting proteins by affinity purification, although the isolated interacting proteins can be immobilized at one position on a solid support, the identities of these interacting proteins may not be known without further identification.

In a variant of the standard interactive protein array, instead of interacting with just one ligand, proteins immobilized at a position may interact with a set of ligands, e.g., protein ligands with a common property, or proteins with a similar structure. For example, four different proteins are immobilized at one position, two of them interact with one protein ligand, and the other two interact with another protein ligand. The two ligands may have a common sequence. In another variant of interactive protein array, the proteins interacting with a ligand may be immobilized at more than one position on the array, e.g., for the purpose of assay control. If immobilized at more than one region, they can be identical or different, e.g., isolated by the same ligand from different sources, such as different tissues.

One important application of interactive protein arrays is to examine protein-protein interactions. For example, to identify what protein ligands interact with a protein of interest, the ligands that potentially interact with the protein of interest are used as affinity baits to prepare their interacting complexes. The number of the ligands should be large enough to contain one or more ligands that truly interact with the protein of interest. The complexes are then immobilized to make an interactive protein array, which has a plurality of positions, each position containing the protein complexes interacting with a ligand. If the protein of interest indeed interacts with a ligand, then it is a component of the protein complexes that interact with the ligand and is present at the position where protein complexes interacting with that ligand are immobilized. Therefore, if the protein of interest is found to be present at one (or several) position, the ligand with which protein complexes immobilized at that position interact is thus identified as being interacting with the protein of interest.

Therefore, several steps are needed to use the above method to screen protein-protein interactions in a protein sample (e.g. a lysate of a cell line, a tissue lysate, or a mixture of lysates from several sources). First, the proteins of interest are selected, of whose interacting ligands are to be identified. Second, the potential interacting ligands of the proteins of interest are chosen. The number of the potential interacting ligands is usually large enough to likely contain one or more real interacting ligands. Third, interactive protein arrays are prepared which include a plurality of positions, each position containing the proteins interacting with one potential interacting ligand. And fourth, the proteins of interest are detected on the interactive arrays to find out which potential interacting ligand interacts with it. When the interactions of more than one protein of interest are to be examined, they can be screened on the same or different arrays prepared from the same protein sample.

The protein of interest can be identified on an interactive protein array by its unique properties, such as its enzymatic activity, or its interactions with other proteins. To facilitate the identification, the protein of interest can be present as a recombinant fusion protein with a detectable tag (such as a fluorescent protein) and then it can be identified by the properties of the tag (e.g., fluorescence, in the case of a fluorescent protein). A widely used method for identifying the protein of interest is to use a specific antibody, in a manner similar to that commonly used in immunoblotting techniques. In one preferred embodiment, the interactive protein array is incubated with antibodies against the protein of interest, long enough to allow efficient association between them. Then the location of the antibodies (as well as the antigen) is identified (e.g. through an enzyme-conjugated secondary antibody). In many applications, enzyme-conjugated primary antibodies are preferred.

Another important application of interactive protein array is to screen modulators (inhibitors or enhancers) of protein-protein interactions. The modulators can be any chemicals: inorganic molecules, organic molecules, oligonucleotides, peptides, and proteins. Small organic chemicals are of particular interest because of their potential use as therapeutic agents. In one embodiment, two protein samples are prepared, one without and one with treatment with the test compound (potential inhibitor or enhancer). If the test compound is an inhibitor of a protein-protein interaction, then the interaction will be disrupted. The protein-protein interactions in the two protein samples are detected by using protein arrays as described. If a protein-protein interaction is decreased or increased, then the compound is found to be an inhibitor or an enhancer of this protein-protein interaction, respectively. The test compound may be present all the time in the assay to ensure its actual effect on protein-protein interactions. One significant advantage of the method is that since many protein-protein interactions can be examined in an assay, the effects of the test compound(s) on a large number of protein-protein interactions can be determined rapidly. Therefore, the method is potentially many times more efficient than other methods of identifying modulators of protein-protein interactions. If a compound is found to be a modulator of a protein-protein interaction, which is associated with or causative of a disease or condition, the compound may be useful for diagnosing, preventing, or treating the disease or condition. A compound identified by the present method as a modulator of a protein-protein interaction can also be used in understanding the physiological functions of that protein-protein interaction.

The first step in making an interactive protein array is to prepare the proteins that are to be immobilized at each position. Since the proteins at each position interact with a specific ligand, the ligand can be used to prepare these interacting proteins. This can be achieved by several methods. In one preferred embodiment, one set of interactive proteins that will be immobilized at a position is prepared and the procedure is repeated to obtain the interactive proteins to be immobilized at all positions.

Co-immunoprecipitation can be used to isolate the interacting partners of a protein ligand. To do so, protein complexes containing the ligand and its interacting partners are first prepared, usually by lysing cells. Then antibodies against the ligand are added to the proteins to allow formation of the immune complex, which contains antibodies, antigens (the ligands), and antigen-interacting proteins. A solid-phase matrix containing protein A (or protein G) is added and the immune complexes are allowed to bind by adsorption of the antibodies to protein A. After the protein A-antibody interaction occurs, the unbound proteins are removed by washing, leaving the purified immune complexes bound to the matrix. When protein complexes are isolated by antibodies, the protein complexes can be arrayed together with antibodies on the solid support. However, if the protein complexes can be separated from the antibodies, it is possible to elute the protein complexes only and immobilize them on a solid support. Separation of protein complexes from antibodies will facilitate the identification of the protein of interest by an antibody in the subsequent applications. There are several methods to separate the protein complexes from the antibodies. In one method, the antibodies are covalently immobilized on a solid support. After immunoprecipitation with the immobilized antibodies, the interacting protein complexes are eluted from the antibodies and solid support under conditions that do not break the covalent bonds.

Affinity purification is a similar method that can be used to isolate proteins interacting with a ligand. In this method, ligands, e.g., recombinant proteins, are first immobilized on beaded material. The beaded material includes cellulose, glass beads, Sephadex, Sepharose, agarose, polyacrylamide, porous particulate alumina, hydroxyalkyl methacrylate gels, diol-bonded silica or porous ceramics. The immobilization can be accomplished covalently or non-covalently, e.g., the immobilization of Glutathione S-transferase (GST) fusion proteins on Glutathione beads. Then the immobilized ligands are used to precipitate interacting partners from a protein solution. After separating the complex of the ligands and their interacting partners from the rest of the proteins in the sample, the complexes are eluted from the solid support. The procedure can be repeated for the isolations of interacting complexes of many ligands. Then the complexes interacting with each ligand are arrayed on a flat solid support at a predetermined position so that the complexes can be identified by the unique position on the support later. In some applications, it is best that only the interacting complexes, but not the ligands, are eluted off the support. This can be done by eluting the interacting proteins under conditions that break up the interactions between the ligands and their interacting proteins but not the interactions between the ligands and the solid support. To facilitate this, ligands can be covalently immobilized on the solid support.

In one preferred embodiment, ligands (recombinant proteins or antibodies) are immobilized on a flat solid support, such as a polyvinylidene difluoride (PVDF) or a nitrocellulose membrane, and used to purify interacting proteins. There are several advantages to immobilize proteins on a membrane support instead of beaded materials. First, the immobilization is easy to do. For most purposes, simple spotting of a ligand solution on a PVDF or a nitrocellulose membrane is satisfactory. Second, the use of a membrane support (e.g. a PVDF membrane with a diameter larger than 1 mm) instead of microscopic beads (usually less than 0.1 mm in diameter) will facilitate the subsequent precipitation steps. Third, it is easy to immobilize proteins at high density on a membrane support, which will facilitate protein-protein interactions.

In another preferred embodiment, the interacting proteins of many ligands are isolated simultaneously. This can be done by first immobilizing the ligands on a flat support to make a protein array, each ligand at a predetermined position, then the array is used to capture the interacting proteins of all the ligands simultaneously. In one preferred embodiment, a set of recombinant protein ligands are prepared and each of them is immobilized on a support at a specific position to form a recombinant protein array. After blocking the array with a saturating amount of non-specific proteins (e.g., bovine serum albumin) to prevent further direct binding of proteins to the support, the protein array is incubated with a protein sample containing proteins that interact with the recombinant protein ligands. During the incubation, each recombinant protein will interact with its interacting proteins and capture them at the same position where the recombinant protein is immobilized. After the capture, non-binding proteins are washed off. Thus, the proteins interacting with the set of recombinant proteins are isolated and arrayed on the support. Proteins interacting with a specific recombinant protein will be localized at the position where the recombinant protein is immobilized.

In another preferred embodiment, antibodies against a set of protein ligands are immobilized on a support to form an antibody array; the antibody against a specific ligand is immobilized at a predefined position. The antibody array is then used to capture the ligands and their interacting proteins from a protein sample, e.g., a cell lysate. To do so, an antibody array is incubated with a protein sample prepared under the conditions that native protein-protein interactions are preserved. After incubation, unbound or non-specifically bound proteins are removed by washing. Therefore, at the position where antibody against a ligand is immobilized, if present in the sample, the ligand and its interacting proteins will be also immobilized at that position. In some situations, when a set of ligands are captured by an antibody array, the ligands may not be captured together with their respective interacting proteins, as not all of the interacting proteins may be present in the protein sample. Therefore, a protein sample containing the interacting proteins is subsequently incubated with the array to allow the interactions to occur between the ligands and their interacting proteins. Several incubations with protein samples may be required before a satisfactory interactive protein array is produced.

Each position of the interactive protein arrays made by the above methods contains protein complexes interacting with a specific ligand or ligands. The immobilization of the protein complexes is via interacting with the ligands, which in turn are immobilized on the solid support via covalent or non-covalent bonds. The immobilization via protein-protein interactions is relatively weak and in many applications adversely limits the use of the arrays. For example, the captured protein complexes may subsequently dissociate from the ligands in the assay, in which case loss of interacting proteins occurs. Therefore, strong immobilization of the complexes on the support is preferred. In the methods described here, in which the interactive proteins are isolated simultaneously with protein arrays (either antibody arrays or recombinant arrays), it is difficult to directly immobilize the interactive proteins on the support. This is because before capturing them, the solid support was saturated with non-specific proteins to prevent any further direct binding of proteins to the support. One way to strongly immobilize the interactive proteins on the support is to covalently cross-link them to the ligands that are used to capture them and that are immobilized on the support via strong covalent or multi-valent non-covalent bonds.

Chemical cross-linking is a process involving the joining of two molecular components by a covalent bond achieved through the use of cross-linkers. Cross-linkers are bifunctional reagents containing two reactive functional groups. The components may be proteins, peptides, drugs, nucleic acids, or solid particles. The two components can be different, e.g., proteins can be cross-linked to non-protein molecules, such as DNA, RNA, and some small organic molecules. Cross-linking can be between two non-interacting molecules, such as the covalent conjugation of enzymes to antibodies (Wong, Shan S., Chemistry of protein conjugation and cross-linking. Boca Raton: CRC Press, 1993). In this case, although the cross-linking efficiency is not high, the cross-linking is generally satisfactory because purified enzymes and antibodies are usually used and no other proteins are present in the cross-linking solutions. Cross-linking can also be between two interacting molecules, such as two interacting proteins. The cross-linking efficiency is much higher between two interacting proteins than that between two non-interacting proteins. However, in most cases, the cross-linking of two interacting proteins is performed in the presence of many other irrelevant proteins whose presences will adversely affect the cross-linking efficiency and produce undesired cross-linking products. Cross-linking has been used in a variety of applications, including studying protein-protein interactions, protein-DNA interactions (Biggin, M. D. Methods Enzymol. 304, 496-515, 1999; Pruss et al., Methods Enzymol., 304, 516-533, 1999), stabilizing protein tertiary structures, determining distances between reactive groups within or between protein subunits, attaching ligands to solid supports, and identifying membrane receptors of known ligands. There are hundreds of known cross-linkers and a variety of methods have been developed to use them to cross-link proteins (Wong, Shan S., Chemistry of protein conjugation and cross-linking. Boca Raton: CRC Press, 1993).

The known cross-linkers and methods can be used in the present invention to immobilize proteins on the solid supports. For example, after an interactive protein array is produced as stated above, the interacting protein complexes can be immobilized on the solid support by covalently cross-linking them with the ligands already strongly immobilized on the support. To do so, the interactive array is incubated with a cross-linking solution for a certain time to allow cross-linkers to react with proteins. A common cross-linking solution that can be used is a formaldehyde solution. A typical formaldehyde solution is 1% formaldehyde in phosphate-buffered saline. The concentration of formaldehyde can be varied and is applicable in many different buffer solutions.

In one preferred embodiment, a recombinant protein array is used to capture proteins interacting with the immobilized recombinant proteins simultaneously and form an interactive protein array. Then the interactive protein array is incubated in a cross-linking solution (e.g. 1% formaldehyde) for a certain time to covalently cross-link the recombinant proteins and their interacting (directly or indirectly) proteins. At the same time, the interacting proteins will be also cross-linked among themselves and thus their interactions are stabilized.

In another preferred embodiment, an antibody array is incubated with a protein sample to capture proteins (antigens) and their interacting partners simultaneously. Then the antigens, interacting partners, and antibodies are covalently cross-linked together to form an interactive protein array. The cross-linking is usually performed after washing off the non-specifically bound proteins. However, in some cases, the cross-linking can be performed before washing. Nonspecific background may occur and needs to be carefully controlled. Under some situations, the antigens and antigen-interacting proteins can be captured by the antibody array sequentially; that is, an antibody array is incubated with a protein sample to capture the antigens first; then the array is incubated with another protein sample to capture the proteins that interact with the antigens. In these cases, after antibodies capture the antigens (with or without antigen-interacting proteins), the antibody-antigen complexes are covalently cross-linked. The array is then incubated with a second protein sample to allow the antigens to bind their interacting proteins present in the second protein sample. Cross-linking is performed again to stabilize the complexes of the antigens and their interacting proteins. In this method, the capture of interacting partners and immobilization of interacting complexes can be repeated many times as deemed appropriate for the assay.

It will be apparent for people familiar with the art that the combination of protein arrays and covalent cross-linking can be used in many other applications, such as detecting protein posttranslational modifications and protein expression profiles. For example, to detect protein posttranslational modifications, an antibody array is first incubated with a protein sample so that the antibodies will capture their specific antigens. After that cross-linking is performed to strongly immobilize the captured antigens on the array. Then the posttranslational modification of these antigens, such as phosphorylation, and glycosylation, can be studied. The use of cross-linking in studying protein posttranslational modifications has similar advantages as those in studying protein-protein interactions.

The present method involves the steps of immobilizing a first molecule (e.g., an antibody or a recombinant protein) on a solid support, association of the first molecule with a second molecule and cross-linking the first and the second molecules. The steps of capturing and cross-linking can be performed multiple times. The cross-linking efficiency is high in the present method for several reasons. First, the proteins to be cross-linked interact with each other and thus are at close proximity. Second, the proteins are at the interface of liquid and solid phase and are at high local concentration. Third, the cross-linking is devoid of interference by other irrelevant proteins.

In addition to the strong immobilization of the interacting proteins on the support, there are several other advantages to using chemical cross-linking in combination with protein arrays. First, covalent cross-linking will simultaneously cross-link the proteins in a complex and stabilize the complex, thus increasing assay sensitivity. The use of interactive protein array in screening protein-protein interactions includes several steps of washes and incubations, which are needed before the production of the final detection signal. Although washing is necessary to avoid nonspecific protein binding, because protein-protein interactions are reversible, washing, especially a long extensive wash, will inevitably disrupt some protein complexes. Even for strong interactions, such as some high-affinity antibody-antigen interactions with an association constant larger than 10⁹ M⁻¹, the half-life time for dissociation of antibody-antigen complexes is in the range of minutes (Sachs et al. Inactivation of staphylococcal nuclease by the binding of antibodies to a distinct antigenic determinant. Biochemistry Nov, 7, 1972; 11(23): 4268-73). The washing and incubation times are long enough to allow efficient dissociation between them. This is especially a problem for many less strong interactions. Therefore, if the dissociation of protein complexes is prevented by covalent cross-linking, the amount of protein complexes bound to the support would be increased, and consequently, the final signal would be enhanced. A second advantage of cross-linking is that it would enable researchers to use a protein array multiple times. Before an array is reused, the previously probed antibody has to be stripped off. The common conditions used to strip proteins render the protein array (particularly the antibody array) unusable because the conditions would denature the proteins (particularly the antibodies) so that they cannot interact with potential partners. After cross-linking, the stripping will not remove the binding partners. Therefore, the array can be re-used without the need to capture interacting partners again. A third advantage is that the cross-linking in the method will save a lot of precious protein samples. After cross-linking, multiple probing requires no new capture of binding partners. Therefore, the method would conserve precious protein samples.

In many immunological methods, after their formation, complexes between antigens and primary antibodies should be maintained until the end of the procedure. However, several factors contribute to the dissociation of antibody-antigen complexes after their formation. For example, in standard Western blotting (immunoblotting), after the formation of antigen-antibody complexes on a membrane, the subsequent extensive washes and incubations, which usually last up to hours, and include several buffer changes, could break up a significant portion of the antigen-antibody complexes. In a preferred embodiment, after binding of the primary antibodies to the antigens, the antigen-antibody complexes on the membrane are stabilized by covalent chemical cross-linking. The cross-linking will prevent the dissociation of antigen-antibody complexes and thus will increase the final detection signal. In an overlay assay, a method related to Western blotting, a protein blot is first incubated with a protein of interest that interacts with one or more proteins on the blot. The blot is then incubated with a primary antibody against the protein of interest and after that, a secondary antibody (usually enzyme-conjugated) is used to locate the primary antibody (therefore, the protein of interest and the proteins it binds). In this method, several protein-protein interactions (the interactions between the protein of interest and its binding proteins, between the protein of interest and its primary antibody, and between primary and secondary antibody) need to be maintained before a final signal is produced. Disruption of any interaction would result in a decreased final detection signal. Therefore, stabilization of these interactions by covalent cross-linking would be very effective in enhancing the detection signal. Other immunological methods, such as immunoprecipitation, ELISA, and immunostaining, employ similar steps as Western blotting (such as the incubation to allow the formation of antibody-antigen complexes, washing to remove non-binding antibodies, etc.), therefore, stabilization of antibody-antigen complexes by covalent cross-linking is also effective in increasing detection signals in these methods.

In some immunoassays, where both direct and indirect protein-protein interactions are required, stabilizing these protein-protein interactions by cross-linking is very effective in enhancing detection signals. For example, in a co-immunoprecipitation assay, in order to find out what proteins may interact with a protein of interest, several interactions have to be maintained: the interaction between the protein of interest and its antibody and the interactions between the protein of interest and a plurality of second proteins. The disruption of any interaction would result in a decreased final signal. Therefore, stabilizing these interactions by covalent cross-linking would be very effective in enhancing signals in these assays. In the assays that involve several protein-protein interactions, cross-linking can be performed repeatedly, at multiple steps.

In another preferred embodiment, an array of antibodies immobilized on a flat support is used to capture the antigens and their interacting proteins. After washing away non-binding proteins, the interacting complexes captured at each position are dissociated from the antibodies and transferred onto a second flat support and immobilized on it. The transfer is performed in such a manner that the positional information is retained; that is, the proteins from a specific position on the first support will be transferred to a specific identifiable position on the second support. Because the interactions between antibodies and the solid support are normally much stronger than the interactions between antibodies and antigens, conditions can be found to disrupt antibody-antigen interactions but leave antibody immobilization on the support intact. Under such conditions, the antigen complexes but not the antibodies can be transferred onto another solid support and immobilized on it.

In a typical transfer, the support containing the antibodies and the interacting protein complexes is placed in contact with a second support. Then they are placed in a buffer solution that could disrupt the interactions between the antigens and antibodies. At the same time, an electric current is applied so that the dissociated proteins will move from the first support to the second support. After completion, the two supports are separated. The transfer and immobilization may happen simultaneously or can be done sequentially. That is, proteins are transferred first and then immobilized. If the immobilization is not as strong as required, the proteins can be immobilized again, e.g., through covalent bonding.

In another preferred embodiment, a plurality of non-antibody ligands such as recombinant proteins, are immobilized on a first solid support by covalent or non-covalent bonds to form a protein array. The array is then incubated with a protein sample to capture the interacting proteins of each ligand. Then the complexes are dissociated and the interacting partners of the ligands are transferred and immobilized onto a second solid support. Conditions can be found so that as little amount of the ligands as possible is transferred to the second solid support.

In another preferred embodiment, after capture by a protein array (an antibody array or a recombinant protein array), the interacting protein complexes are dissociated from the solid support and the proteins in the complexes are separated from each other, e.g., by SDS/PAGE, and then transferred to a second support. The positional information is preserved during the procedure. To facilitate the separation by electrophoresis, a one-dimensional array can be used.

There are many advantages to the method of transferring interacting protein complexes to a second support to form an interactive protein array. First, after transfer, it is easy to immobilize the interactive proteins on the second support. Second, when using a regular antibody array to study protein-protein interactions, HRP-conjugated primary antibodies have to be used to detect the protein of interest. By using the present method to separate the protein complexes from the capturing antibodies and transfer them to another support, the common procedure for western blot can be used (i.e., first blotting with an un-conjugated primary antibody and then with a secondary antibody conjugated to an enzyme or a fluorescent molecule). Sometimes the primary antibodies immobilized on the first support may also be transferred to the second support together with the interacting protein complexes and it is therefore difficult to use enzyme-conjugated secondary antibodies. A third advantage of the method is that protein complexes can be easily and strongly immobilized on the support after transfer. Therefore, after examining a protein with its specific antibody on the array, the antibody can be stripped off the array without affecting the transferred proteins. Therefore, the array can be probed with another antibody for another protein. In fact, the membrane can be probed multiple times. Another advantage of using this method is that the protein complexes on a protein array can be transferred to multiple membrane supports and thus multiple interactive protein arrays can be produced rapidly.

The present invention is described here, primarily for the use in examining protein-protein interactions. It will be obvious for those skilled in the art that the methods and their modified versions may be applied in examining the interactions between proteins and molecules other than proteins as well. In one embodiment, an oligo array is incubated with a protein sample to allow the binding between proteins and immobilized oligos. Then the oligo-protein complexes are stabilized by covalent cross-linking. A protein of interest can be examined to see if it is captured by the oligo array and if it is, at what position it is captured. From this information, whether the protein of interest interacts with DNA can be answered. The invention can also be used to detect the interactions between non-protein molecules. These molecules include but are not limited to DNAs, RNAs, oligos, lipids, carbohydrates, and small organic chemicals. In addition, the methods described in the present invention will also be useful in many other applications, which are only limited by the imagination of users.

### EXAMPLES

The following examples are offered by way of illustration only and are by no means intended to limit the scope of the claimed invention.

### Example 1

### Making Interactive Protein Arrays by Isolating Interactive Proteins Sequentially

Twenty antibodies against different proteins were used in this example to immunoprecipitate their respective antigens from a lysate of COS7 cells. The lysate was made under conditions to preserve the native protein-protein interactions. Each of the twenty antibodies (about 2.5 micrograms) was incubated with the cell lysate separately overnight at 4° C. Then protein A agarose beads were added to precipitate antibody-antigen complexes. Under this condition, the proteins interacting with the antigens were also precipitated. After washing with PBS, the precipitates were eluted from the beads by incubating with 10 ul of 2% SDS in PBS. Two microliter of each elution was immobilized on a PVDF membrane, each at a specific position, to form an interactive protein array.

### Example 2

### Use of Interactive Protein Array to Screen Protein-Protein Interactions

The interactive protein array (made in Example 1) was used to screen protein-protein interactions. In this example, STAT1 was used as the protein of interest and probed on the array. STAT1 was identified at several positions and thus it was found to interact with several proteins, including FADD, TRADD, beta-catenin and NF-KB.

### Example 3

### Use of Antibodies Immobilized on PVDF Membranes to Isolate Interacting Protein Complexes Sequentially

Two micrograms of anti-E-cadherin antibodies were immobilized on a PVDF membrane disc (2 mm in diameter). After blocking with non-fat dry milk (1% in PBS) for 1 hour, the membrane was used to capture E-cadherin and its associated proteins from a lysate made from a rat liver cell line (1% Triton soluble fraction). The membrane disc was put inside a 1.5 ml eppendorf tube and incubated with the lysate for 2 hrs, after that the disc was washed with 1% Triton in PBS for three times. Then the proteins captured by the antibodies were eluted by incubating with 10 ul of 2% SDS in PBS. The same procedure was repeated for nineteen other antibodies to isolate their antigens and antigen-interacting proteins. Then 2 ul of each elution was immobilized on a PVDF membrane, each at a pre-defined position to form an interactive protein array.

The interactive protein array was used to screen protein-protein interactions. In this example, Beta-catenin was used as the protein of interest and probed on the array. It was found at the positions where E-cadherin and its interacting proteins were immobilized, agreeing with the fact that beta-catenin interacts with E-cadherin.

### Example 4

### Making Interactive Protein Arrays by Isolating Interactive Proteins Simultaneously

In this example, an interactive protein array was made by isolating the interactive proteins simultaneously. Twenty different antibodies (0.5 microgram each) were immobilized on a PVDF membrane to form an antibody array, each antibody at a predefined position. The antibody array was then incubated with a COS7 cell lysate to capture the antigens and their interacting proteins. After incubation, unbound or non-specific bound proteins were removed by washing. Therefore, at the position where an antibody was immobilized, its antigen and the antigen-interacting proteins were also immobilized at that position.

The interactive protein array was used to screen protein-protein interactions. In this example (FIG. 2A), STAT1 was used as the protein of interest and probed on the array. STAT1 was found in several positions and thus it interacted with several proteins.

### Example 5

### Cross-Linking Antibody-Antigen Complex Enhances Western Blotting Signal

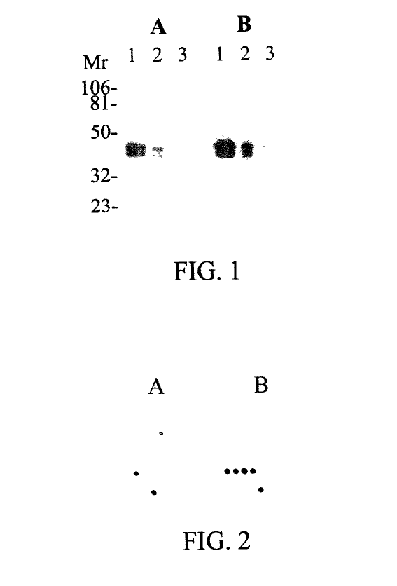

Total tissue lysate from mouse heart was subjected to SDS/PAGE separation (FIG. 1). After transfer to the PVDF membrane, the blot was probed with anti-connexin43 antibodies. One half of the amount of protein loaded in the first lane was loaded in the second lane; and one fourth was loaded in the third lane. A: western blotting with anti-connexin43 antibodies without cross-linking; B: western blotting with anti-connexin43 antibodies with cross-linking by formaldehyde. 2% Glutaraldehyde and a homobifunctional N-hydroxysuccimide ester cross-linker, Bis (Sulfosuccinimidyl) suberate (BS, from Pierce, Rockford, Ill.; with a final concentration of 2 mM) were equally effective. The cross-linking was performed at room temperature for 1 hour.

### Example 6

### Cross-Linking Antibodies, Antigens, and Antigen-Interacting Proteins Enhances the Signal in Screening Protein-Protein Interactions with Antibody Arrays

Two identical interactive protein arrays were made as described in Example 4. Array A was not treated while Array B was treated with 2% formaldehyde for 1 hr to cross-link the protein complexes on the array. The arrays were then incubated with horseradish peroxidase-conjugated STAT1 antibody followed by enhanced chemiluminescence (ECL) detection. STAT1 was detected at several positions on both arrays but the ECL signal was stronger on Array B (FIG. 2B) than on Array A (FIG. 2A).

### Example 7

### Cross-Linking with Formaldehyde and Probing for Two Different Proteins

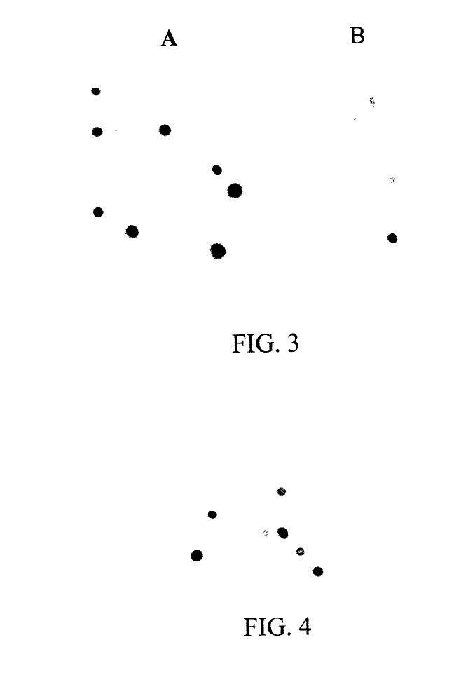

An interactive protein array made as described in Example 4 was treated with 2% formaldehyde for 1 hr to cross-link protein complexes on the array. Then the array was incubated with HRP-conjugated antibody against STAT1 (FIG. 3A). After identifying STAT1 on the array, STAT1 antibody was stripped from the array by incubating in 100 mM glycine pH 2.5 plus 100 mM beta-mercaptanol ethanol for 30 min. After the stripping, it could be determined that the antibody against STAT1 was removed from the array. Then the same array was probed with another antibody, HRP-conjugated antibody against beta-catenin. Beta-catenin was found at several positions (FIG. 3B).

### Example 8

### Making of Interactive Protein Arrays by Transferring

Fifty antibodies against different proteins were arrayed on a solid support. After capturing the respective antigens and antigen-interacting proteins, the bound antigens and their interacting proteins were separated from the antibodies, transferred onto a second solid support and immobilized on it. The transferred proteins could then be detected. In this example, STAT1 protein was detected on the interactive array by STAT1 specific antibodies (FIG. 4). The antibodies could be immobilized on the solid support by covalent binding so that they would not be transferred.

What is claimed is:

1. An array of biological reagents immobilized on a solid support, said array having separate known sites, said reagents at each said site interacting directly or indirectly with one or more ligands, and said reagents being covalently cross-linked to said ligands.

2. The array of claim 1, wherein said array has between 100 and 100,000 sites

3. The array of claim 1, wherein said ligands are antibodies.

4. The array of claim 1, wherein said ligands are recombinant proteins.

5. The array of claim 1, wherein said ligands are purified proteins.

6. The array of claim 1, wherein said ligands are nucleic acids.

7. The array of claim 1, wherein said ligands are oligo nucleotides.

8. A method of making an array of biological reagents, comprising the steps of
immobilizing a plurality of ligands on a solid support, each said ligand at a separate known site;
contacting said ligands on said solid support with a plurality of said reagents to allow the binding between said ligands and said reagents;
covalently cross-linking said ligands and said reagents.

9. The method of claim 8, wherein said ligands are antibodies.

10. The method of claim 8, wherein said ligands are recombinant proteins.

11. A method of making an array of biological reagents, comprising the steps of
immobilizing a plurality of ligands on a first solid support, each said ligand at a separate known site;
contacting said ligands on said solid support with a plurality of said reagents to allow the binding between said ligands and said reagents;
transferring and immobilizing said reagents bound to said ligands onto a second solid support.

12. The method of claim 11, wherein said ligands are covalently immobilized on said first solid support.

13. The method of claim 11, wherein said reagents are non-covalently immobilized on said second solid support.

14. The method of claim 11, wherein said ligands are antibodies.

15. The method of claim 11, wherein said ligands are recombinant proteins.

16. A method of making an array of biological reagents on a solid support, comprising the steps of
preparing said reagents by using ligands that bind said reagents;
immobilizing said reagents on said solid support.

17. The method of claim 16, wherein said ligands are antibodies.

18. The method of claim 16, wherein said ligands are recombinant proteins.

19. The method of claim 16, wherein said ligands are immobilized on membranes made of materials selected from the group consisting of nitrocellulose, nylon, polyvinylidene difluoride, glass, or plastics, and their derivatives.

20. A method of biological assays, comprising the steps of:
immobilizing a plurality of ligands on a solid support, each said ligand at a separate know site;
incubating said ligands on said solid support with a plurality of reagents to allow the binding between said ligands and said reagents;
covalently cross-linking said ligands and said reagents.

21. The method of claim 20, wherein one side of said support is at least 1 millimeter long.

22. The method of claim 20, wherein said ligands are antibodies.

23. The method of claim 20, wherein the number of said ligands ranges from 1 to 1,000,000.

24. The method of claim 20, wherein the number of said ligands ranges from 100 to 10,000.

25. A kit useful for detecting proteins, the kit containing a solid support, on which a plurality of reagents are immobilized, each at a predetermined position, said reagents at each said position interacting with one or a plurality of ligands.

26. The kit of claim 25, wherein said reagents are proteins.

27. The kit of claim 25, wherein said ligands are antibodies.

28. The kit of claim 25, wherein said ligands are recombinant proteins.

29. The kit of claim 25, wherein said reagents are covalently cross-linked with said ligands.
